(19) United States
(12) Patent Application Publication
(10) Pub. No.: US 2019/0184084 A1
(43) Pub. Date: Jun. 20, 2019

(54) DIALYZER AND FABRICATING METHOD THEREOF

(71) Applicants: LITE-ON ELECTRONICS (GUANGZHOU) LIMITED, Guangzhou (CN); Lite-On Technology Corporation, Taipei (TW)

(72) Inventors: Wei-Ming Shieh, Taipei (TW); Yi-Feng Pu, Taipei (TW); Pei-Hsuan Huang, Taipei (TW)

(73) Assignees: LITE-ON ELECTRONICS (GUANGZHOU) LIMITED, GUANGZHOU (CN); Lite-On Technology Corporation, Taipei (TW)

(21) Appl. No.: 16/219,964

(22) Filed: Dec. 14, 2018

(30) Foreign Application Priority Data
Dec. 18, 2017 (CN) 201711362497.1

(51) Int. Cl. A61M 1/16

(52) U.S. Cl. CPC A61M 1/1623; A61M 1/1654; A61M 1/1652; A61M 1/1601

(57) ABSTRACT

A dialyzer and a fabricating method thereof are provided. The dialyzer includes a housing, a hydrophilic layer, a fixing layer, a plurality of hollow fiber membranes, and two end caps. The housing has a first opening and a second opening, and is provided with a dialysate inlet and a dialysate outlet, wherein an entire peripheral surface of the housing located between the first opening and the dialysate inlet is a first portion, and an entire peripheral surface of the housing located between the second opening and the dialysate outlet is a second portion. The hydrophilic layer is disposed on the inner wall of the first portion and the second portion, wherein the hydrophilic layer and the housing are different materials. The fixing layer is disposed on the hydrophilic layer and fixes the hollow fiber membranes to the inner wall of the housing.

### CROSS-REFERENCE TO RELATED APPLICATION

This application claims the priority benefit of China application serial no. 201711362497.1, filed on Dec. 18, 2017. The entirety of the above-mentioned patent application is hereby incorporated by reference herein and made a part of this specification.

### BACKGROUND OF THE INVENTION

### Field of the Invention

The invention relates to a blood treatment device and a fabricating method thereof, and more particularly, to a dialyzer and a fabricating method thereof.

### Description of Related Art

Patients of renal failure cannot discharge body wastes such as protein-digested products, urea, creatinine, phosphate, and vitamin B12, and therefore require dialysis to compensate for the natural excretory function of the kidneys. A common dialysis includes, for instance, purifying the blood of a patient using a dialyzer to remove excess water and toxins from the blood.

The materials of the housing applied in a dialyzer are mostly polyvinyl chloride (PVC), polycarbonate (PC), polypropylene (PP), polysulfone (PSU), and polyethylene terephthalate (PET), etc, wherein PVC contains halogen, and PET and PC decompose to form toxic dioctyl phthalate and bisphenol A (BPA). In addition, many dialyzers face the problem of the poor compatibility between the housing material and the potting material.

### SUMMARY OF THE INVENTION

The invention provides a dialyzer and a fabricating method of the same, facilitating good bonding and compatibility between the housing and the potting material used in the dialyzer.

The dialyzer of the invention includes a housing, a hydrophilic layer, a fixing layer, a plurality of hollow fiber membranes, and two end caps. The housing has a first opening and a second opening opposite to each other, wherein a first portion of the housing is arranged between the first opening and a dialysate inlet, and a second portion of the housing is arranged between the second opening and a dialysate outlet. The hydrophilic layer is disposed on the inner wall of the housing corresponding to the first portion and the second portion, wherein the hydrophilic layer and the housing are different materials. A plurality of hollow fiber membranes are disposed in the housing. The fixing layer is disposed on the hydrophilic layer for fixing the hollow fiber membranes to the inner wall of the housing. Two end caps are respectively disposed at two ends of the housing.

In an embodiment of the invention, a groove is disposed in the first portion and the second portion, and the hydrophilic layer is disposed in the groove.

In an embodiment of the invention, the surface roughness of the inner wall of the first portion and the second portion is, for instance, 0.1 micrometer (μm) to 1.5 mm.

In an embodiment of the invention, the housing and the hydrophilic layer are, for instance, integrally formed.

In an embodiment of the invention, a material of the hydrophilic layer may include a hydrophilic resin having a hydrophilic functional group.

In an embodiment of the invention, the hydrophilic functional group can include —COOH, —COOR, —COR, —R₁OR₂, —Ar—O—R, —Ar₁—O—Ar₂, —ROH, —R₁SO₂R₂, —RCONH₂, —NH, —CONR, —TiO, —SiO, —COOM, or Ca₁₀⁺(PO₄)₆(OH)₂⁻, wherein each of R, R₁, and R₂ is independently a hydrocarbon group, each of Ar, Ar₁, and Ar₂ is independently an aryl group, and M is a metal.

In an embodiment of the invention, the hydrophilic resin may be, for instance, polymethylmethacrylate (PMMA), polysulfone (PSU), or polyamide (PA).

In an embodiment of the invention, the hydrophilic resin may have a hydrophobic end.

In an embodiment of the invention, the material of the housing is, for instance, polypropylene, polybutene (PB), polyethylene (PE), or a combination thereof.

In an embodiment of the invention, the dialyzer may further comprise a melting join layer disposed between the hydrophilic layer and the housing.

The fabricating method of the dialyzer of the invention includes the following steps. First, a hydrophilic layer is formed on an inner wall of a housing, wherein the housing has a first opening and a second opening opposite to each other, and the hydrophilic layer and the housing are different materials. Next, a plurality of hollow fiber membranes are placed in the housing. Next, a fixing layer is formed on the hydrophilic layer to fix the hollow fiber membranes onto the inner wall of the housing. Next, two ends are respectively disposed on the first opening and the second opening.

In an embodiment of the invention, the inner wall of the housing may include a groove, and the hydrophilic layer is formed in the groove.

In an embodiment of the invention, the inner wall of the housing has a rough surface, and the hydrophilic layer is formed on the rough surface, wherein the surface roughness of the rough surface is, for instance, 0.1 μm to 1.5 mm.

In an embodiment of the invention, forming the hydrophilic layer on the inner wall of the housing is, for instance, double injection molding to integrally form the hydrophilic layer and the housing.

In an embodiment of the invention, the material of the hydrophilic layer is, for instance, a hydrophilic resin having a hydrophilic functional group.

In an embodiment of the invention, the hydrophilic functional group is, for instance, —COOH, —COOR, —COR, —R₁OR₂, —Ar—O—R, —Ar₁—O—Ar₂, —ROH, —R₁SO₂R₂, —RCONH₂, —NH, —CONR, —TiO, —SiO, —COOM, or Ca₁₀⁺(PO₄)₆(OH)₂⁻, wherein each of R, R₁, and R₂ is independently a hydrocarbon group, each of Ar, Ar₁, and Ar₂ is independently an aryl group, and M is a metal.

In an embodiment of the invention, the hydrophilic resin is, for instance, polymethylmethacrylate (PMMA), polysulfone (PSU), or polyamide (PA).

In an embodiment of the invention, the hydrophilic resin may have a hydrophobic end.

In an embodiment of the invention, the material of the housing is, for instance, polypropylene, polybutene (PB), polyethylene (PE), or a combination thereof.

In an embodiment of the invention, forming the fixing layer on the hydrophilic layer includes the following. Temporary caps are disposed at the two ends of the housing. A fixing layer material is injected in the housing. A centrifugation process is performed to fill the fixing layer material in the first opening and the second opening. The fixing layer material is cured to form the fixing layer, wherein at least a portion of the fixing layer is in contact with the hydrophilic layer. The temporary caps are moved.

Based on the above, a hydrophilic layer is formed on the inner wall of the two ends of the housing of the dialyzer of the invention, and the hydrophilic layer may increase the surface energy of the inner wall of the two ends of the housing, and therefore bonding with the hydrophilic fixing layer can be facilitated.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic of a dialyzer of an embodiment of the invention.

FIG. 2 is a partial enlarged view of the dialyzer of FIG. 1.

FIG. 3 is a partial enlarged view of a dialyzer of another embodiment of the invention.

FIG. 4 is a flowchart of a fabricating method of a dialyzer of an embodiment of the invention.

### DESCRIPTION OF THE EMBODIMENTS

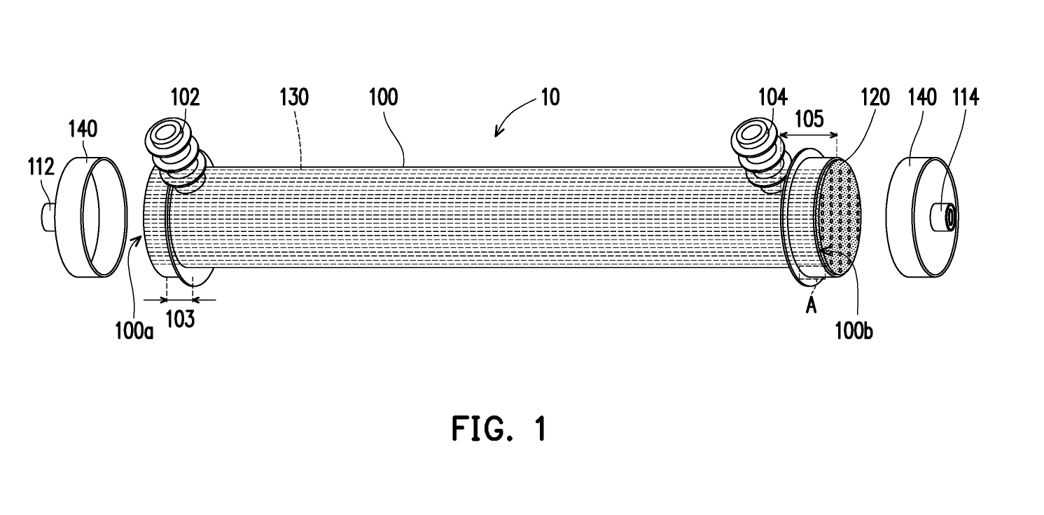

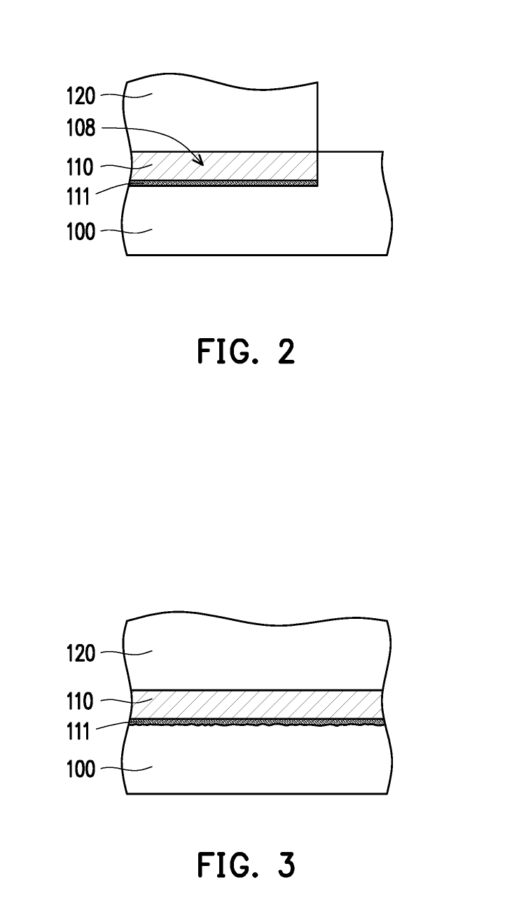

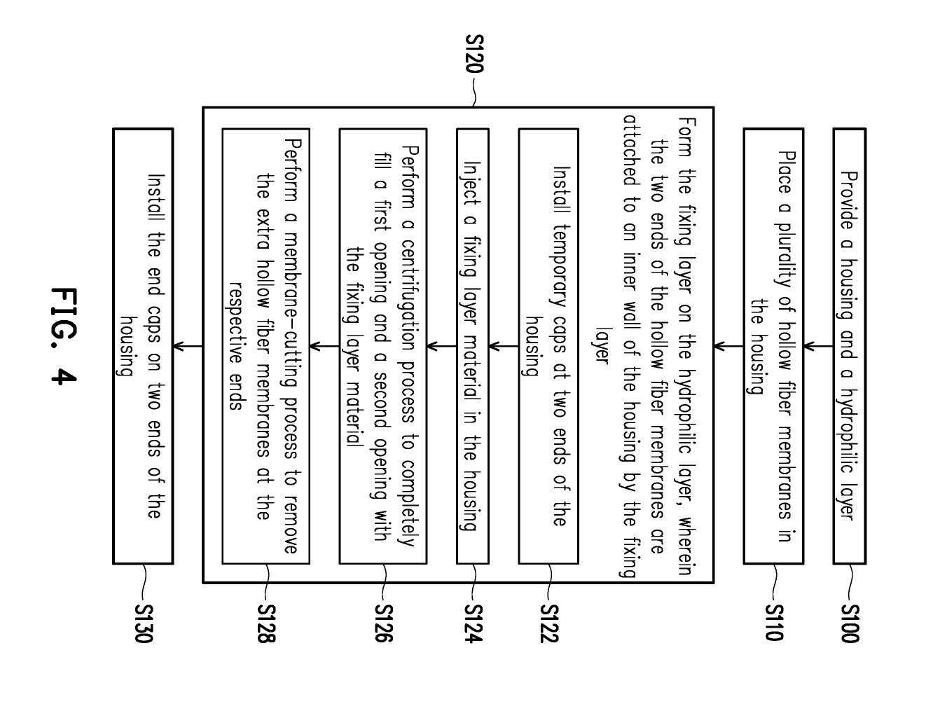

FIG. 1 is a schematic of a dialyzer of an embodiment of the invention. FIG. 2 and FIG. 3 are partial enlarged views of region A of a dialyzer 10 of FIG. 1. Although FIG. 2 and FIG. 3 respectively show enlarged configuration of one end of the dialyzer, region A of FIG. 2 and FIG. 3 can be applied to the other end as needed. For clarity and better understanding, a hydrophilic layer is not shown in FIG. 1, and hollow fiber membranes are not shown in FIG. 2 and FIG. 3.

Referring to FIG. 1 and FIG. 2, the dialyzer 10 includes a housing 100, a hydrophilic layer 110, a fixing layer 120, a plurality of hollow fiber membranes 130, and two end caps 140. The housing 100 is, for instance, a hollow tubular structure to accommodate the hollow fiber membranes 130. The housing 100 is, for instance, made of a hydrophobic material. In the present embodiment, the material of the housing 100 is, for instance but not limited thereto, polypropylene, polybutylene, polyethylene, or a combination thereof. In another embodiment, other hydrophobic materials may also be used as the material of the housing 100. The housing 100 has a first opening 100a and a second opening 100b opposite to each other, and is provided with a dialysate inlet 102 and a dialysate outlet 104. In an embodiment, the dialysate inlet 102 is close to the first opening 100a, and the dialysate outlet 104 is close to the second opening 100b.

In an embodiment, the housing 100 includes a first portion 103 and a second portion 105. Specifically, the entire peripheral section of the housing 100 located between the first opening 100a and the dialysate inlet 102 is defined as the first portion 103, and the entire peripheral section of the housing 100 located between the second opening 100b and the dialysate outlet 104 is defined as the second portion 105.

In the present embodiment, the hydrophilic layer 110 is disposed on the inner wall of the housing 100 corresponding to the first portion 103 and the second portion 105. The hydrophilic layer 110 and the housing 100 are different materials. Specifically, the host material of the hydrophilic layer 110 and the host material of the housing 100 are substantially different. In an embodiment, the monomer forming the hydrophilic layer 110 is different from the monomer forming the housing 100. The material of the hydrophilic layer 110 is, for instance, a hydrophilic resin having at least one hydrophilic functional group. In an embodiment, the hydrophilic functional group contained in the hydrophilic resin is, for instance, —COOH, —COOR, —COR, —R₁OR₂, —Ar—O—R, —Ar₁—O—Ar₂, —ROH, —R₁SO₂R₂, —RCONH₂, —NH, —CONR, —TiO, —SiO, —COOM, or Ca₁₀⁺(PO₄)₆(OH)₂⁻, wherein each of R, R₁, and R₂ is independently a hydrocarbon group, each of Ar, Ar₁, and Ar₂ is independently an aryl group, and M is a metal. However, the invention is not limited thereto, and the hydrophilic functional group of the hydrophilic resin may be selected from other suitable hydrophilic functional groups. In an embodiment, R₁ and R₂ can be the same or different, and Ar₁ and Ar₂ can be the same or different. In an embodiment, —R₁SO₂R₂ is a functional group corresponding to polysulfone. In the present embodiment, the hydrophilic resin has a hydrophobic end and a hydrophilic end, wherein the hydrophilic end includes the hydrophilic functional group described above, and the hydrophobic end is, for instance, a long-chain hydrocarbon. The number of carbon atoms in the long-chain hydrocarbon of the hydrophobic end and the weight-average molecular weight of the hydrophilic resin are not particularly limited. The hydrophilic resin is, for instance, polymethylmethacrylate (PMMA), polysulfone (PSU), or polyamide (PA). In an embodiment, the hydrophilic resin can be polymethylmethacrylate, wherein the hydrophilic functional group thereof is —COOH, and the hydrophobic end thereof is —CH.

Since the hydrophilic layer 110 is formed on the inner wall of the housing 100 corresponding to both of the first portion 103 and the second portion 105, the hydrophobic end of the hydrophilic resin may have good bonding with the hydrophobic housing 100, and the hydrophilic end of the hydrophilic resin can increase the surface energy of the inner wall of the first portion 103 and the second portion 105, thereby facilitating the bonding of the hydrophilic fixing layer 120.

Referring to FIG. 2, the first portion 103 of the housing 100 includes a groove 108, and the groove 108 and the dialysate inlet 102 can be spaced apart by a predetermined distance. The second portion 105 of the housing 100 also has a groove 108, and the groove 108 and the dialysate outlet 104 can be spaced apart by a predetermined distance (not shown). In an embodiment, the groove 108 is, for instance, a stepped groove or other structures relatively concave, as long as the thickness of the housing 100 at the groove 108 is less than the thickness of the housing 100 elsewhere. In the present embodiment, the groove 108 can be continuously extended to the entire peripheral surface of the first portion 103 or the second portion 105. In another embodiment, a plurality of grooves 108 are separately distributed over the first portion 103 or the second portion 105, but the invention is not limited thereto. The width of the groove 108 along the longitudinal direction of the housing 100 is, for instance, 0.5 μm to 10 mm. The location, shape, and quantity of the groove 108 depicted in the above embodiment are only for reference, and the invention is not limited thereto, and the arrangement and configuration of the grooves can be adjusted based on process requirements.

Since the first portion 103 and the second portion 105 are provided with the groove 108, the hydrophilic layer 110 can be better fixed in and bonded to the groove 108. Moreover, the hydrophobic end of the hydrophilic resin in the hydrophilic layer 110 can be bonded with the hydrophobic surface of the groove 108, and the exposed hydrophilic surface of the hydrophilic layer 110 would tend to well bond with the hydrophilic fixing layer 120.

FIG. 3 describes the position relationship of the housing 100 and the hydrophilic layer 110 in the dialyzer of another embodiment of the invention. The difference between the embodiment of FIG. 3 and the embodiment of FIG. 2 is that, the first portion 103 of the housing 100 in FIG. 2 includes a groove 108, and the first portion 103 of the housing 100 in FIG. 3 does not include a groove. In the present embodiment, the inner wall of the first portion 103 of FIG. 3 is a rough or patterned surface. Similarly, the inner wall of the second portion 105 could be a rough or patterned surface. Specifically, the surface roughness of the inner wall of the first portion 103 and the second portion 105 is 0.1 μm to 1.5 mm. When the surface roughness of the inner wall of the first portion 103 and the second portion 105 is within the range above, the bonding area of the hydrophilic layer 110 between the first portion 103 and the second portion 105 can be increased. Moreover, the hydrophobic end of the hydrophilic resin in the hydrophilic layer 110 can be bonded with the hydrophobic surface of the first portion 103 and the second portion 105, and the bonding between the hydrophilic surface of the hydrophilic layer 110 and the hydrophilic fixing layer 120 can be enhanced as well.

Similarly, the groove 108 of the first portion 103 and the second portion 105, illustrated in the embodiment shown in FIG. 2, could further incorporate a rough surface with a surface roughness of 0.1 μm to 1.5 mm, such that its bonding with the hydrophilic layer 110 can be further improved.

It should be mentioned that, the housing 100 and the hydrophilic layer 110 having different materials can be integrally formed via double injection molding. The double injection molding includes conducting two injection molding steps in a single mold to fabricate the housing 100 and the hydrophilic layer 110 respectively. Specifically, an injection molding step can be performed first to form the housing 100, and then another injection molding step is performed to form the hydrophilic layer 110. Alternatively, an injection molding step can be performed first to form the hydrophilic layer 110, and then another injection molding step is performed to form the housing 100. Thus, a melting join layer 111 could be formed at the heterojunction between the housing 100 and the hydrophilic layer 110 (shown in FIG. 2 and FIG. 3). In an embodiment, the melting join layer 111 includes the material of the housing 100, the material of the hydrophilic layer 110, or a mixture thereof. The melting join layer 111, for instance, combines the housing 100 and the hydrophilic layer 110 via the viscosity of at least one of the molten materials or chemical bonding, thereby forming an integrated and integral structure. That is, the hydrophilic layer 110 is directly formed and configured on the housing 100 with the melting join layer 111 intervening therebetween. It should be mentioned that, when the inner wall of the housing 100 has a groove or a rough surface, the resulting hydrophilic layer 110 is filled in the recess of the groove or the rough surface, such that the integrally formed surfaces of the housing 100 and the hydrophilic layer 110 are coplanar.

Moreover, the material matching of the double injection molding should be suitably taken into consideration. In an embodiment, the material used in the first injection molding needs to have a higher softening point or melting temperature than the material used in the second injection molding. Otherwise, the melt flushing or wash-out would occur, such that the product profile formed in the first injection molding is deformed. In an embodiment, the hardness of the material used in the first injection molding is higher than the hardness of the material used in the second injection molding. In an embodiment, the shrinkage of each material used in the double injection is between 0.2% and 5%, and the shrinkage difference between the materials respectively used in the first injection molding and the second injection molding is 0% to 4.8%. The above shrinkage is obtained by the size difference between the mold cavity and the molded product at room temperature, which is then divided by the size of the mold cavity. The shrinkage is defined by the thermal expansion and contraction and molding conditions of the materials themselves. During the double injection molding process, the material of the first injection molding and the material of the second injection molding sequentially undergo respective molding. When the materials used in the double injection molding are chosen to have a greater difference in shrinkage, the interfacial strength of the materials would be reduced and the molding product would therefore become to warp. In an embodiment, the difference between the shrinkage of the material used in the first injection molding and the shrinkage of the material used in the second injection molding is about 1%. In another embodiment, the difference between the shrinkage of the material used in the first injection molding and the shrinkage of the material used in the second injection molding is about 0.6%. In an embodiment, the difference between the shrinkage of the material used in the first injection molding and the shrinkage of the material used in the second injection molding is about 0.4%.

Referring to FIG. 1 to FIG. 3, the fixing layer 120 is disposed on the hydrophilic layer 110 and filled in the first opening 100a and the second opening 100b. The material of the fixing layer 120 is, for instance, a hydrophilic material such as polyurethane (PU). In an embodiment, the fixing layer 120 is disposed on the inner wall of the housing 100 at the two ends, and at least a portion of the fixing layer 120 is in contact with the hydrophilic layer 110 of the first portion 103 or the second portion 105 such that the housing 100 and the fixing layer 120 have good bonding properties.

The plurality of hollow fiber membranes 130 disposed in the housing 100 are fixed by the fixing layer 120. The hollow fiber membranes 130 are provided with permeaselectivity and could be semi-permeable membranes. The material of the hollow fiber membranes 130 is, for instance, cellulose acetate, polysulfone (PSU), polyethersulfone (PES), or polymethylmethacrylate (PMMA). In the present embodiment, to increase the compatibility of the hollow fiber membranes 130 with the human body, the material of the hollow fiber membranes 130 can further contain a hydrophilic polymer in addition to the above components. The hydrophilic polymer is, for instance, poly(vinyl pyrrolidone) (PVP), poly(ethylene glycol) (PEG), poly(vinyl alcohol) (PVA), poly(ethylene oxide) (PEO), poly(ethylenimine) (PEI), or poly(acrylate) (PAA). In the present embodiment, the hollow fiber membranes 130 is prepared, for instance, by a dry-wet spinning process. The invention is not limited to the exemplary hollow fiber membranes 130 shown in FIG. 1, and thus the quantity of the hollow fiber membranes 130 can be adjusted as needed. In an embodiment, about 7000 to 12000 hollow fiber membranes 130 can be arranged in the housing 100.

The end caps 140 are respectively disposed at two ends of the housing 100, wherein the two end caps 140 are respectively provided with a blood outlet 112 and a blood inlet 114. In the present embodiment, the dialysate inlet 102 is disposed close to the blood outlet 112 and the dialysate outlet 104 is disposed close to the blood inlet 114, such that the direction of the blood flow is opposite to that of the dialysate flow in the tube so as to achieve a better dialysis effect.

FIG. 4 is a flowchart of a fabricating method of a dialyzer of an embodiment of the invention. Referring to FIG. 1, FIG. 2, and FIG. 4, the following fabricating method will be described with reference to the dialyzer shown in FIG. 1 and FIG. 2.

Step S100 is performed to provide a housing 100 and a hydrophilic layer 110. In the present embodiment, the housing 100 and the hydrophilic layer 110 can be integrally formed via double injection molding. The integrated structure of the housing 100 and the hydrophilic layer 110 has been described in detail in the embodiments above and is therefore not repeated hereafter.

Step S110 is performed to place a plurality of hollow fiber membranes 130 in the housing 100. In an embodiment, since the hollow fiber membranes 130 might be longer than the housing 100, the two ends of the hollow fiber membranes 130 partially extend from the edge of the housing 100.

Step S120 is performed to form the fixing layer 120 on the hydrophilic layer 110, wherein the two ends of the hollow fiber membranes 130 are attached to the inner wall of the housing 100 by the fixing layer 120. In an embodiment, sub-steps S122, S124, S126, and S128 are included in Step S120.

Sub-step S122 is performed to install temporary caps (not shown) at two ends of the housing 100. In this step, the temporary caps can be directly in contact with the two ends of the hollow fiber membranes 130. It is noted that the temporary caps used in the potting process are not provided with blood outlets and inlets, and should not be construed as the end caps of the dialyzer.

Sub-step S124 is performed to inject a fixing layer material, e.g., potting compounds, into the housing 100. Specifically, the fixing layer material (not shown) is injected into the housing 100 via a dialysate inlet and a dialysate outlet. The injected fixing layer material is a hydrophilic material, such as polyurethane.

In sub-step S126, a centrifugation process is performed to fill the fixing layer material in the first opening 100a and the second opening 100b. Specifically, during the centrifugation process, the fixing layer material is evenly distributed at the two ends of the housing 100, and therefore, the first opening 100a and the second opening 100b are filled with and sealed by the fixing layer material. During the centrifugation process, the fixing layer material could be cured to form the fixing layer 120 in this step. The fixing layer material is cured by, for instance, heat curing, UV/infrared curing, moisture curing, or a combination thereof.

Since the hydrophilic layer 110 is formed on the inner wall of the housing 100 at the two ends (e.g., corresponding to the first portion 103 and the second portion 105), wherein the hydrophilic resin made of the hydrophilic layer 110 has at least one hydrophilic functional group, and therefore its good bonding with the hydrophilic fixing layer 120 can be achieved. Moreover, the housing 100 is configured to include a specific groove or rough surface on the inner wall of the first portion 103 and the second portion 105, thereby increasing the contact area for the hydrophilic layer 110 so as to effectively fix the hydrophilic layer 110 thereon.

In sub-step S128, a membrane-cutting process is performed to remove the extra hollow fiber membranes 130 at the respective ends. Specifically, after the fixing layer 120 is cured and attached to the housing 100, the temporary caps are removed from the two ends of the housing 100, and a portion of the fixing layer 120 and the hollow fiber membranes 130 is then cut off and removed at the respective ends. In an embodiment, after the membrane-cutting process, the fixing layer 120 and the hollow fiber membranes 130 can be protruded from the two end surfaces of the housing 100.

Step S130 is performed to install the end caps 140 on the two ends of the housing 100. In an embodiment, the method of installing the end caps 140 on the two ends of the housing 100 includes placing a sealing element at the two ends of the housing 100 and then fixing the end caps 140 to the two ends of the housing 100, wherein the sealing element could be an o-ring that can increase liquid tightness. In another embodiment, the method of installing the end caps 140 on the two ends of the housing 100 includes welding the end caps 140 to the two ends of the housing 100 via ultrasonic welding. At this point, the dialyzer according to an embodiment of the invention is complete. The dialyzer fabricated above can be further sterilized by, for instance, ethylene oxide sterilization, y-ray sterilization, or steam sterilization.

In the following, examples of the invention are provided to more specifically describe the invention. However, the scope of the invention should not be construed to the following examples, and the exemplary materials and processes, etc. can be modified.

### EXAMPLES

### Fabrication of Hollow Fiber Membrane

A spinning solution was prepared, including 20 wt % of polysulfone (main component), 10 wt % of polyvinylpyrrolidone (hydrophilic polymer), and 70 wt % of N-methylpyrrolidone (solvent). The hollow fiber membranes were prepared using a dry-wet spinning method. Specifically, the spinning solution was discharged from a double-ring nozzle via liquid injection molding (non-coagulation), and the discharged spinning solution was immersed in water, as a non-solvent through a predetermined air gap. After coagulation, washing with a non-solvent, and drying, about 9000 hollow fiber membranes were obtained.

### Fabrication of Housing and Hydrophilic Layer

The housing and the hydrophilic layer located on the inner wall of the housing were fabricated via double injection molding. The material of the housing is injectable medical-grade polypropylene with the melting point of about 150° C. to 160° C. The material of the hydrophilic layer is injectable medical-grade polymethylmethacrylate with the melting point of about 130° C. to 140° C. Specifically, a first injection step was performed to form the polypropylene in a mold. After filling, holding pressure, cooling, and molding, the mold was opened, and the semifinished product remained in the mold. Next, a second injection step was performed to completely fill the cavity of the mold with the polymethylmethacrylate, and then demolding was performed to obtain an integrally-formed housing having a hydrophilic layer firmly attached thereonto.

### Fabrication of End Cap

The end caps were fabricated using an injection molding method using injectable medical-grade polypropylene with the melting point of about 150° C. to 160° C.

### Packaging and Sterilization of Dialyzer

The hollow fiber membranes were placed in the housing via automation equipment. After temporary caps were installed at two ends of the housing, the fixing layer material (polyurethane) was injected into the housing, which is then centrifuged and cured. After removing the temporary caps and conducting the membrane-cutting, the end caps were put in place. Next, ultrasonic welding and sterilization were performed.

Based on the above, the hydrophilic layer is formed on the inner wall of the housing of the dialyzer. As the hydrophilic layer can increase the surface energy of the inner wall of the two ends of the housing, the interactions at material boundaries could be strengthen, thereby facilitating good bonding between the hydrophobic housing and the hydrophilic fixing layer via the arrangement of the hydrophilic layer.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A dialyzer, comprising:
a housing having a first opening and a second opening opposite to each other, wherein a first portion of the housing is arranged between the first opening and a dialysate inlet, and a second portion of the housing is arranged between the second opening and a dialysate outlet;
a hydrophilic layer disposed on an inner wall of the housing corresponding to the first portion and the second portion, wherein the hydrophilic layer and the housing are different materials;
a plurality of hollow fiber membranes disposed in the housing;
a fixing layer disposed on the hydrophilic layer for fixing the hollow fiber membranes to the inner wall of the housing; and
two end caps respectively disposed at two ends of the housing.

2. The dialyzer of claim 1, wherein a groove is disposed in the first portion and the second portion, and the hydrophilic layer is disposed in the groove.

3. The dialyzer of claim 1, wherein a surface roughness of the inner wall of the first portion and the second portion is 0.1 μm to 1.5 mm.

4. The dialyzer of claim 1, wherein the housing and the hydrophilic layer are integrally formed.

5. The dialyzer of claim 1, wherein a material of the hydrophilic layer comprises a hydrophilic resin having a hydrophilic functional group.

6. The dialyzer of claim 5, wherein the hydrophilic functional group comprises —COOH, —COOR, —COR, —R₁OR₂, —Ar₁—O—R₂, —Ar—O—Ar, —ROH, —R₁SO₂R₂, —RCONH₂, —NH, —CONR, —TiO, —SiO, —COOM, or Ca₁₀⁺(PO₄)₆(OH)₂⁻, wherein each of R, R₁, and R₂ is independently a hydrocarbon group, each of Ar, Ar₁, and Ar₂ is independently an aryl group, and M is a metal.

7. The dialyzer of claim 5, wherein the hydrophilic resin comprises polymethylmethacrylate, polysulfone, or polyamide.

8. The dialyzer of claim 5, wherein the hydrophilic resin has a hydrophobic end.

9. The dialyzer of claim 1, wherein a material of the housing comprises polypropylene, polybutylene, polyethylene, or a combination thereof.

10. The dialyzer of claim 1, further comprising a melting join layer disposed between the hydrophilic layer and the housing.

11. A fabricating method of a dialyzer, comprising:
forming a hydrophilic layer on an inner wall of a housing, wherein the housing has a first opening and a second opening opposite to each other, and the hydrophilic layer and the housing are different materials;
placing a plurality of hollow fiber membranes in the housing;
forming a fixing layer on the hydrophilic layer to fix the hollow fiber membranes onto the inner wall of the housing; and
disposing two end caps respectively on the first opening and the second opening.

12. The fabricating method of claim 11, wherein the inner wall of the housing comprises a groove, and the hydrophilic layer is formed in the groove.

13. The fabricating method of claim 11, wherein the inner wall of the housing has a rough surface, and the hydrophilic layer is formed on the rough surface, wherein a surface roughness of the rough surface is 0.1 μm to 1.5 mm.

14. The fabricating method of claim 11, wherein forming the hydrophilic layer on the inner wall of the housing comprises double injection molding to integrally form the hydrophilic layer and the housing.

15. The fabricating method of claim 11, wherein a material of the hydrophilic layer comprises a hydrophilic resin having a hydrophilic functional group.

16. The fabricating method of claim 15, wherein the hydrophilic functional group comprises —COOH, —COOR, —COR, —R₁OR₂, —Ar—O—R, —Ar₁—O—Ar₂, —ROH, —R₁SO₂R₂, —RCONH₂, —NH, —CONR, —TiO, —SiO, —COOM, and Ca₁₀⁺(PO₄)₆(OH)₂⁻, wherein each of R, R₁, and R₂ is independently a hydrocarbon group, each of Ar, Ar₁, and Ar₂ is independently an aryl group, and M is a metal.

17. The fabricating method of claim 15, wherein the hydrophilic resin comprises polymethylmethacrylate, polysulfone, or polyamide.

18. The fabricating method of claim 15, wherein the hydrophilic resin has a hydrophobic end.

19. The fabricating method of claim 11, wherein a material of the housing comprises polypropylene, polybutylene, polyethylene, or a combination thereof.

20. The fabricating method of claim 11, wherein forming the fixing layer on the hydrophilic layer comprises:
disposing temporary caps at two ends of the housing;
injecting a fixing layer material into the housing; and
performing a centrifugation process to fill the fixing layer material in the first opening and the second opening;
curing the fixing layer material to form the fixing layer, wherein at least a portion of the fixing layer is in contact with the hydrophilic layer; and
removing the temporary caps.
